(19) United States
(12) Patent Application Publication
(10) Pub. No.: US 2003/0135170 A1
(43) Pub. Date: Jul. 17, 2003

(54) Precision aid

(76) Inventor: Howard Kapple, Bettendorf, IA (US)

Correspondence Address: Howard Kapple, 1927 Fairmeadows Dr., Bettendorf, IA, 52722, US

(21) Appl. No.: 10/046,045

(22) Filed: Jan. 16, 2002

(51) Int. Cl. A61M 35/00

(52) U.S. Cl. 604/300

(57) ABSTRACT

This device is mounted on a familiar instrument, the eye glass frame. For positioning to service the eye it is precise every time you put them on. A key slot is mounted to the underside of the brow bar above each eye. The key slots are angled outward in front of the brow bar so that the tip of the eye drop bottle clears the eye safely. The key slots are centered exactly in the center of the eye top to bottom and side to side. The key slots are made of copper wire. The shape of the key slots are triangular at the top to funnel down the eye drop bottle into the key slot where the bottle will rest until the drop is delivered. The bottom half of the glass frame and glass was removed and discarded. This was to provide room for one hand to pull down the lower eye lid while the other hand delivers drops from the bottle.

### BACKGROUND

EZY CARE DROP AID has a cup that fits over the eye to deliver eye drops. The other end of this cup has a port that the eye drop bottle fits in. The drops must go clear through the cup to be delivered into the eye. The interior of the cup of course retains a residual amount of the medication meant for the eye. Also the cup would have to be sanitized constantly to avoid contamination. With the eye covered over, one would wonder if the drop ever reached the eye and how many drops reached the eye.

The EYE DROP GUIDE is a plastic cradle that the eye drop bottle fits in. The cradle has a pointed projection under the nipple of the bottle that is placed on the cheek bone below the eye and pressed downward to pull the bottom eyelid down as you squeeze the drop into the eye. As you can see, this has a safety hazard of poking yourself in the eye with the projected point while searching for the cheekbone.

FIRST DROP is another cradle device that the drop bottle fits into and comes in three different sizes for different size bottles. It also requires seeking out bone features of the face to locate the eye. I think perhaps the safety of this invention is also lacking. It depends too much on a person wandering around the face trying to locate the proper features around the eye.

### SUMMARY

A device for delivery of the eye drops to the center of the eye all by yourself It is as fast and easy as putting on a pair of glasses. Just put the eye drop in the key slot and wait for the drop as you hold your head back. It puts the drop in the center of the eye directly without touching anything else along the way eliminating the chance of contamination and infection. It also insures that you get the full amount of medication of each drop into the eye. Keeping the key slot of Precision Aid clean is as easy as caring for your eye glasses, Just wash them in soap and water by hand.

### DESCRIPTION OF THE PREFERRED DOCUMENT

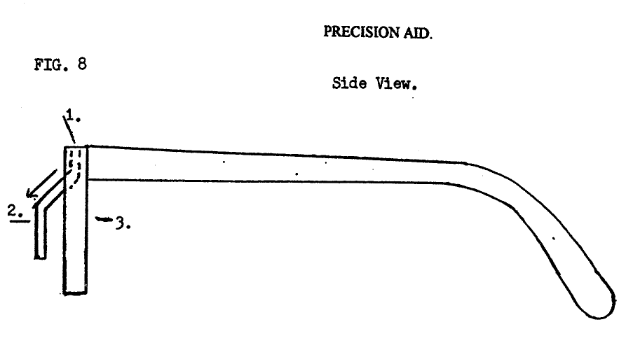

Precision aid is a device for giving yourself eye drops in the center of the eye every time. Because eye glasses are so precise in their service to the eyes as to positioning over the eyes I decided they would be perfect for mounting my Precision Aid device also. I cut away the bottom half of the glass frame and discarded the glass. This gave room for one hand to pull down the lower eye lid while the other hand delivers the drop. I used 5/64″ copper wire to make what I call a key slot assembly, one for each eye. The slot at the bottom of the keyslot assembly where the eye drop bottle rests is ¼″ wide to fit the nipple of the bottle. Once the bottle is in the slot it can not move about while waiting for the drop. The key slot assembly is 1″ wide at the top and is triangular shaped to funnel downward the eye drop bottle into the ¼″ slot rest. The key slot assembly part #2 page 1 FIG. 6 of the prints are shown anchored to the bottom of the brow bar #1 page 1 FIG. 6 of the frame. These key slot assemblies are angled outward in front of the brow bar ¼″ to add to the normal clearance of the glass frame to the eyes giving at least a 112″ safety clearance of the tip of the eye drop bottle to the eyes. From the bottom of the brow bar to the inside bottom of the key slot where the eye drop bottle rests is 6/8″. This positions the eye drop bottle over the center of the eye top to bottom. To center the bottle left to right over the eye I measure from the center of the nose saddle #3 print page 1 FIG. 6 shown as letter C out to the left and to the right 1-¼″ to the center of the key slot assemblies #2 page 1 FIG. 6 also shown with a letter C. Print page 4 FIG. I shows a template 1 use to locate where to drill the four 5/64″ holes to mount the key slot assemblies # 2 FIG. 6 to the bottom of the brow bar #1 also Shown on FIG. 6. I line up the C on the template with the C on the nose saddle and mark the location of the holes on the bottom of the brow bar. The holes are drilled almost all the way through the bottom side of the brow bar. The copper key slot assemblies are made of 5/64″ wire and the holes are of the same size to assure a tight fit. Print page 2 and 3 show FIG. 7 and FIG. 8 the ¼″ protrusion of the key slot assemblies in front of the brow bar.

### MAKING THE KEY SLOT ASSEMBLIES

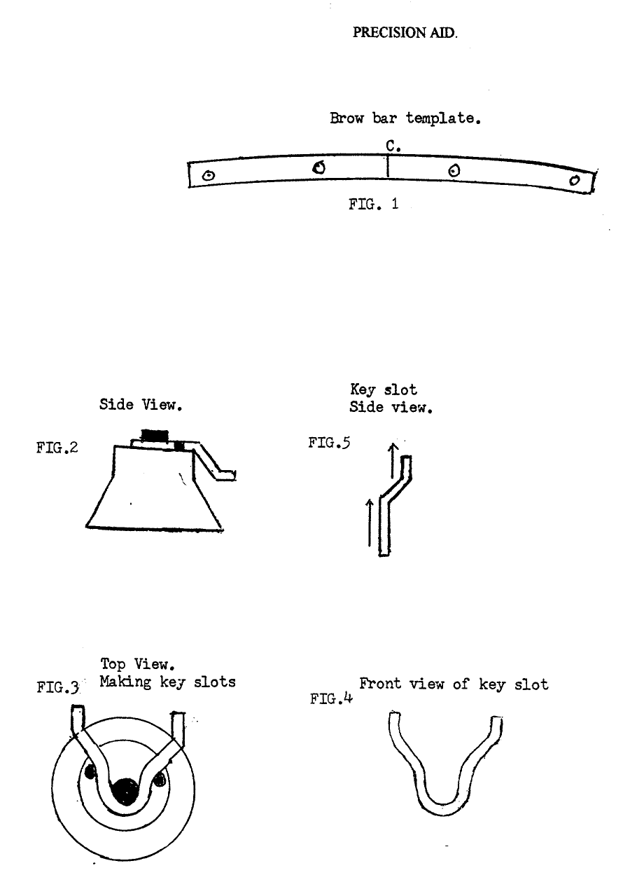

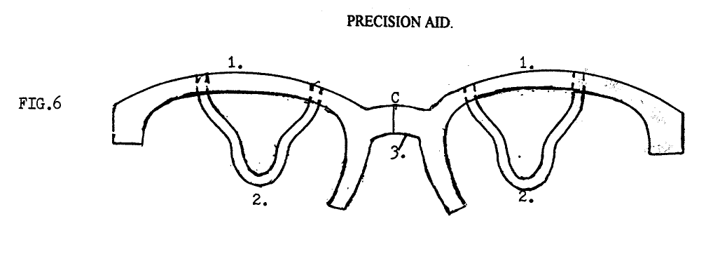

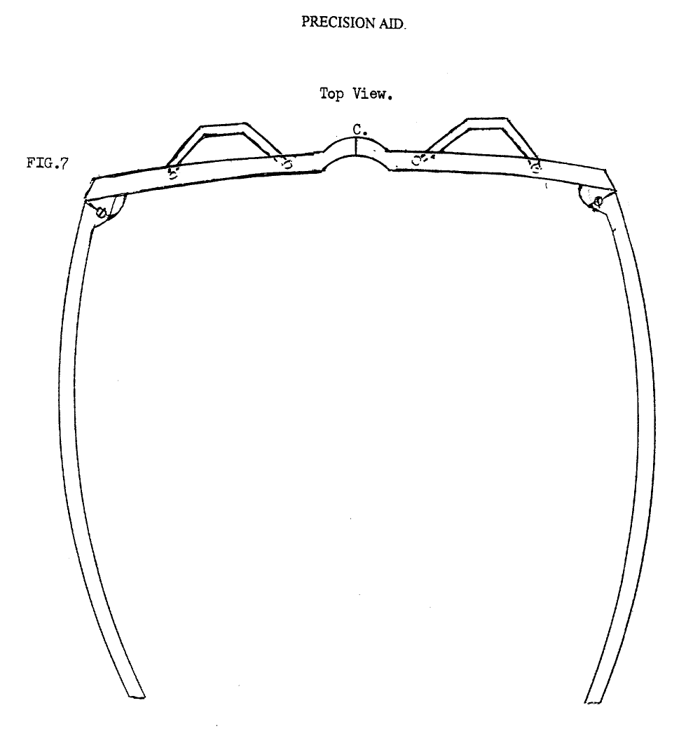

The key slot assemblies are made on a bench die block as shown by print page 4 FIG. 2, FIG. 3, FIG. 4 & FIG. 5 made from copper wire 2-⅜″ long.

### SPECIFICATIONS ON HOW TO USE

First remove the cap from the eye drop bottle and wipe off the nipple of the bottle with a fresh unmedicated paper tissue like Puffs. The bottle may have fell over and in this case the nipple would be wet. Do this before and after every drop so that none of the medication gets on the key slots. Keep the key slots clean by washing with soap and water.

Put on the PRECISION AID device and tilt head back to receive the drops into the eye.

Close the eye not receiving the drop so you can see the key slot to put the eye drop bottle in for the eye receiving the drop.

If you look down at the tip of the eye drop bottle delivering the drop you will see the drop go into the eye.

Using PRECISION AID is fast and easy and the regimen of giving yourself 4 and 5 drops a day is no longer a dread. You also know that the full medication of each drop is getting into the center of the eye.

1. Precision Aid key slots are designed to funnel downward the tip of the eye drop bottle into the slot be held securely over the center of 5the drop is delivered.

2. Precision Aid key slot units are also designed to protrude in front of the brow bar of the glass frame ¼″ so the tip of the eye drop bottle has a safety clearance above the eye of ½″ and the slot holds the bottle steady until the drop is deliver without contamination.

3. Precision Aid key slot units are designated to be anchored into the most precise positioning instrument ever invented to service the eyes consistently. The drops are delivered to the center of the eye without directly touching anything, eliminating any chance of contamination and infection.
